(19) United States
(12) Patent Application Publication
(10) Pub. No.: US 2022/0001105 A1
(43) Pub. Date: Jan. 6, 2022

(54) DETACHABLE SENSOR DEVICES

(71) Applicant: EZMEMS LTD., Netanya (IL)

(72) Inventors: Tsvi SHMILOVICH, Pardes Hanna-Karkur (IL); Nicola MOLINAZZI, Kfar Sava (IL)

(21) Appl. No.: 17/292,653

(22) PCT Filed: Nov. 10, 2019
(86) PCT No.: PCT/IL2019/051223

§ 371 Date: May 10, 2021

Related U.S. Application Data
(60) Provisional application No. 62/758,562, filed on Nov. 10, 2018.

(51) Int. Cl. A61M 5/168; A61M 5/14

(52) U.S. Cl. CPC A61M 5/16804; A61M 5/1413; A61M 2205/3317; A61M 2205/3561; A61M 2205/3334; A61M 2205/50; A61M 2205/3368; A61M 2205/3306; A61M 2205/3375; A61M 2209/088; A61M 2205/3324

(57) ABSTRACT

Disclosed a monitoring device comprising housing configured to attach to a user or to a user aid item, at least one attachment assembly provided in the housing and configured to attach to a respective at least one removable sensor module, electric circuitries or contacts mounted in or on the housing and configured to provide an electrical interface between the at least one removable sensor module and one or more external devices, and inlet and outlet fasteners provide in the housing and configured to receive and hold respective fluid supply and fluid dispensing lines. The fluid supply and fluid dispensing lines can be connected to the at least one removable sensor module attached to the at least attachment assembly for dispensing fluid media therethrough. The at least one sensor module can be configured to measure at least one property or condition of the fluid media, and the electrical circuitry can be configured to communicate measurement data or signals generated by the at least one sensor module to the one or more external devices.

### TECHNOLOGICAL FIELD

The present invention is generally in the field of sensor devices, and in particular relates to configurations of detachable sensor devices.

### BACKGROUND

Small size sensors e.g., Silicon based pressure sensors, usually employ sensor elements implemented by semiconductor structures, which are not suitable for direct integration into fluidic devices and/or appliances.

In the age of sensors and Internet of Things (IoT), where sensors are everywhere and everything is being sensed, the deployment of a large number of sensors in plastics (e.g., for medical devices, pharma/medicine dispensing, determination of quality of food/agriculture products, industry equipment/machinery, and for packaging) requires use of inexpensive, small size, and sometimes and disposable, sensor devices. Plastics items nowadays lack sensing capabilities because Silicon based microelectromechanical systems (MEMS) cannot be integrated into plastic products without dramatically increasing the costs, and also because it will limit their capabilities.

The present application provides smart arrangements for the encapsulation of sensors into devices and appliances.

For medical applications, the Internet of Medical Things (IoMT) can help monitor, inform and notify with actual data to identify issues before they become critical and allow earlier intervention. On the other hand, there is a constant demand for solutions to lower healthcare costs.

For example, most of hospitalized patients undergo infusion drug therapy, but no direct measurement of the drug delivery leads to alarm fatigues and adverse drug events (ADEs). Alarm fatigues leads to expensive time spent to verify the infusion integrity and silence false alarms. ADEs injuries are very common, especially in intensive care unit (ICU), emergency room (ER) and during night time. Although smart infusion pumps were introduced more than a decade ago, infusion safety issues are a top food and drug administration (FDA) priority because the infusion set, which connects the pumps to the patient, is the ‘weak link’ which is most likely to fail.

Some solutions known from the patent literature are briefly described hereinbelow.

US Patent Publication No. 2012/290268 describes a modular sensor assembly in which a sensing module may be packaged and provided separately from a signal processing module and which, in some applications, may facilitate disposal and/or replacement of the sensing module when exposed to a dirty or contaminated environment without requiring disposal and/or replacement of the entire sensor assembly. In certain applications, the sensing module may include at least one transducer or sensor and a local memory containing a set of conditioning coefficients. The sensing module may be removably coupled to a signal processing module which, in some cases, may be configured to download the set of conditioning coefficients stored in the local memory of the sensing module, and to use the set of conditioning coefficients to produce a substantially linearized output signal.

US Patent Publication No. 2016/305797 describes a sensor apparatus that may include a base module having a first interface configured to receive a signal from at least one sensor module. The base module may also include a controller coupled to the first interface and configured to process the signal to produce data, and may include a second interface configured to communicate the data to a computing device.

International Patent Publication No. WO 2018/100176 describes a disposable and removable cartridge configured to cooperate with a wearable support device, said wearable support device comprising means to be secured to the skin of a subject, at least one connection track and at least one first positioning means; said disposable and removable cartridge comprising: at least one microneedle; a sensing area; a microfluidic circuit in fluid communication with said at least one microneedle and with said sensing area; at least one sensor adapted to monitor at least one parameter of a fluid providing from the patient, in the sensing area; a second connection track electrically connected to the at least one sensor; at least one second positioning means configured to cooperate with the at least one first positioning means of the support device in such a way that the at least one first connection track of the support device is connected at said at least one second connection track of the disposable and removable cartridge, and in such a way the cartridge is releasably secured to the wearable support device.

### General Description

The present application provides fluidic sensor devices configured to utilize removable/replaceable, and in some embodiments also disposable, sensor modules (also referred to herein as sensor chips). The sensor modules disclosed herein generally comprise at least one inlet port configured to reversibly connect to a respective at least one fluid supply vessel/conduit, at least one fluid outlet port configured to reversibly connected to a respective at least one fluid dispensing vessel/conduit, at least one fluid passage configured to connect between the at least one fluid inlet port and the at least one fluid outlet port, and at least one sensor element. The sensor module is configured to pass a stream of fluid media received from the at least one fluid supply vessel/conduit, measure one or more parameters, properties and/or conditions (generally referred to herein as fluid properties), of the received fluid media by means of the at least one sensor element, and discharge the received fluid media through the at least one fluid outlet port.

The sensor module is configured in some embodiments for placement in a monitoring device (also referred to herein as acquisition unit). The monitoring device comprising one or more sockets, each configured to receive and hold at least one sensor module, and at least one fastening channel configured to receive and hold the at least one fluid inlet/outlet port of the sensor module and/or its respective at least one fluid supply/dispensing vessel/conduit. In some embodiment the monitoring device comprises at least one inlet fastening channel configured to receive and hold the at least one fluid inlet port of the sensor module, and/or its respective at least one fluid supply vessel/conduit, and at least one outlet fastening channel configured to receive and hold the at least one fluid outlet port of the sensor module, and/or its respective at least one fluid dispensing vessel/conduit.

In some embodiments the monitoring device further comprises electrical contacts and/or circuitries configured to communicate data/signals with the at least one sensor module placed in the at least one socket of the device, and process/analyze and/or display data indicative of the data/signals received from the at least one sensor module. The circuitries provided in the monitoring device may be further configured to transmit (wirelessly and/or over conducting wires) the data/signals to one or more external computer devices and/or data networks for processing/analysis, storage and/or display.

The sensor modules, with, or without, the monitoring devices, can be used in a myriad of various different applications wherein properties of a dispensed fluid are needed to be measured continuously, periodically or intermittently. For example, and without being limiting, infusion systems are typically connected to a patient by disposable lines/tubes, which do not offer fluid sensing capabilities. Therefore, conventional infusion systems can only estimate the pressure and flow rate of the dispensed fluid, but they don't and cannot measure these properties directly within the infusion line/tube. Direct measurement of pressure and/or flow within the disposable line/tube is essential to ensure safety. The integration of disposable pressure and/or flow sensors is a promising solution to ensure smart, reliable and safe infusion systems.

The present application, in some embodiments, is directed to a polymeric and/or plastic sensor module configured to measure one or more properties of a fluid streamed therethrough e.g., pressure and/or flow rate and/or temperature and/or conductivity and/or optical properties. More particularly, but not exclusively, the sensor module also includes all the sensing elements, and the connectivity modules (mechanical, electrical and data/signals communication) with external devices. The sensor module can be configured to be plugged or inserted into a reusable monitoring device, which includes all the reusable elements e.g., for communication with external systems and/or to power the sensor device. The sensor module can include fluid channels or structures used to perform the measurement, or to allow additional functionality (e.g., liquid passage, pressure release, mechanical connectivity, and suchlike).

Accordingly, in some of the embodiments disclosed herein, a sensor apparatus is provided that comprises a disposable sensor module and a reusable housing/monitoring device configured to receive and hold the disposable sensor module, and communicate data/signals with one or more sensor elements of the sensor module. The ability to differentiate between the disposable and reusable elements of the sensor apparatus disclosed herein enables, inter alia, a dramatic cost reduction, compact size, easy integration into plastic products, robustness, ability to perform in different types of environments, easy customization, fast go to market, and/or ability to sense multiple parameters with the same device. The embodiments disclosed herein provide all of these features with excellent measurement characteristics, similar to high-end semiconductor based sensor devices currently available in the market (e.g., Silicon based sensors).

Dramatic cost reduction can be achieved with the sensor apparatus configurations disclosed herein due to the innovative manufacturing approach of the sensor modules, which eliminates the need of packaging costs (70-90%, or more, of the costs of the conventional sensor devices). In addition, the integration process of the sensor module into plastic/polymeric products and introducing it into working environments is much simple and reduces the implementation costs.

The sensor devices/elements used in the different embodiments disclosed herein can be implemented using, but not limited to, the techniques and embodiments disclosed in international patent publication Nos. WO 2018/235087, titled “Sensor elements on thin foils/films”, WO 2018/092130, Titled “High resistance strain gauges and methods of production thereof”, and WO 2018/025264, titles “Fluidic microelectromechanical sensors/devices and fabrication methods thereof”, which are all of the same applicant hereof, and the disclosure of which is incorporated herein by referenced.

One inventive aspect disclosed herein relates to a monitoring device comprising housing configured to attach to a user (e.g., as a wrist-wear device) or to a user aid item, at least one attachment assembly provided in the housing and configured to attach to a respective at least one removable sensor module, electric circuitries or contacts mounted in or on the housing and configured to provide an electrical interface between the at least one removable sensor module and one or more external devices, and inlet and outlet fasteners provide in the housing and configured to receive and hold respective fluid supply and fluid dispensing lines. The fluid supply and fluid dispensing lines can be connected to the at least one removable sensor module attached to the at least attachment assembly for dispensing fluid media therethrough. The at least one sensor module can be configured to measure at least one property or condition of the fluid media. The electrical circuitry can be configured to communicate measurement data or signals generated by the at least one sensor module to said one or more external devices. Optionally, the electric circuitries are configured to wirelessly communicate the measurement data or signals to the one or more external devices.

The monitoring device may comprise one or more processors configured and operable to process the measurement data or signals generated by the one or more sensor modules and generate control data or instructions for a flow control unit for controlling flow rate of the fluid media. A display device may be used in the monitoring device, so the one or more processors can display in the display device information associated with the measurement data or signals.

In some embodiment the fluid media comprises a medicament dispensed into the user. At least one of the sensor modules comprise in some embodiments electrodes configured to conduct the dispensed fluid media and measure one or more electrical properties thereof.

The one or more sensor modules can be configured to measure one or more of the following: fluid pressure; flow rate; temperature of the dispensed fluid media and/or of the user; electrical conductivity and/or resistance, dielectric constant and/or dissipation factor; pH level; optical transparency and/or transmission; and/or acoustic signals transferred through the dispensed fluid media. Optionally, but in some embodiments preferably, the control unit is configured and operable to process the measurement data/signals from the one or more sensor modules and identify based thereon one or more substances comprised in the dispensed fluid media.

A measurement arrangement is arranged in some embodiments using the monitoring device of the embodiments disclosed herein and the one or more sensor modules removably attached to the attachment assembly. At least one of the one or more sensor modules may comprise at least one sensor element made of polymeric or plastic materials and having one more transducing elements formed thereon or therein. Optionally, at least one of the one or more sensor modules is a disposable sensor module.

In some embodiment at least one sensor element comprises electrical contacts electrically connected to the one more transducing elements, and the one or more transducing elements and their electrical contacts are formed on, or at least partially in, a deformable element. Optionally, the deformable element is made of an integrated multilayered foil or film. The at least one external layer of the multilayered foil or film can be configured to enable attachment to an injected molded plastic for assembly in a body of the sensor module.

In some embodiments the one or more transducing elements are configured to form at least one differential pressure measurement circuitry. The housing and/or at least one of the one or more sensor modules may comprise optical elements configure to measure at least one optical property of the dispensed fluid media.

The measurement arrangement may further comprise a flow control device configured to controllably adjust flow rate of the dispensed fluid media. In some embodiment the control unit is configured and operable to generate control signals for adjusting the flow rate of the dispensed fluid media based on the measurement data/signals from at least one of the one or more sensor modules.

Another inventive aspect disclosed herein relates to a method of measuring one or more properties of a dispensed fluid media. The method comprising connecting at least one fluid supply conduit to inlet port of a respective at least one sensor module, where the at least sensor module having a fluid passage in fluid communication with the inlet port and one or more sensor elements configured to measure one or more properties of fluid media in the fluid passage, electrically coupling between the at least one sensor module and an acquisition unit, where the acquisition unit is configured to provide an interface to the at least one sensor module, and processing data/signals generated by the at least one sensor module and obtained via the acquisition unit.

The method comprises in some embodiments communicating the measurement data/signals from the acquisition unit to at least one external device. Optionally, the at least one external device comprises a flow control unit. The method may thus comprise generating control signals/data based on the measurement data/signals for regulating flow of the dispensed fluid media by the flow control unit.

The method comprises in some embodiments processing the measurement data/signals and identifying at least one substance contained in the dispensed fluid media. In some embodiments the method comprises attaching the at least one sensor module to the acquisition unit or to a user receiving the fluid media.

### BRIEF DESCRIPTION OF THE DRAWINGS

In order to understand the invention and to see how it may be carried out in practice, embodiments will now be described, by way of non-limiting example only, with reference to the accompanying drawings. Features shown in the drawings are meant to be illustrative of only some embodiments of the invention, unless otherwise implicitly indicated. In the drawings like reference numerals are used to indicate corresponding parts, and in which:

FIG. 1 schematically illustrates use of a wearable monitoring device, and its connectivity to IoMT environments, according to some possible embodiments;

FIG. 2A to FIG. 2C schematically illustrate use of sensors integrated into a needle hub for fluid dispensing applications according to some possible embodiments;

FIG. 3 exemplifies shows a conventional fluid infusion system;

FIGS. 4A and 4B schematically illustrate use of the wearable monitoring device for fluid dispensing applications;

FIGS. 5A and 5B schematically illustrate use of the wearable monitoring device for fluid infusion according to some possible embodiments;

FIG. 6 schematically illustrates a removable sensor module/chip usable in the wearable monitoring device according to some possible embodiments;

FIGS. 7A to 7C schematically illustrate a wearable monitoring device according to some possible embodiments;

FIGS. 8A to 8D schematically illustrate applications of the wearable monitoring device on a body of a user/patient according to some possible embodiments;

FIG. 9 schematically illustrate various communication and/or electrical units that can be used in the sensor module according to some possible embodiments;

FIGS. 10A to 10D schematically illustrate implementation of the wearable monitoring device according to some possible embodiments in a form of watch;

FIGS. 11A and 11B schematically illustrate sectional view of a wearable monitoring device according to some possible embodiments; and

FIGS. 12A and 12B schematically illustrates possible connector designs usable to connect to the sensor module according to some possible embodiments.

### DETAILED DESCRIPTION OF EMBODIMENTS

One or more specific embodiments of the present disclosure will be described below with reference to the drawings, which are to be considered in all aspects as illustrative only and not restrictive in any manner. In an effort to provide a concise description of these embodiments, not all features of an actual implementation are described in the specification. Elements illustrated in the drawings are not necessarily to scale, or in correct proportional relationships, which are not critical. Emphasis instead being placed upon clearly illustrating the principles of the invention such that persons skilled in the art will be able to make and use the devices/apparatuses, once they understand the principles of the subject matter disclosed herein. This invention may be provided in other specific forms and embodiments without departing from the essential characteristics described herein.

There is a need to integrate sensor(s) into plastic appliances in a compact, simple and cost effective way. The present application provides configurations and fabrication techniques to integrate plastic based sensor(s) in the form of a plastic chip directly into plastic products/devices and applications, where the integration of the sensor(s) is direct and without requiring packaging preparations before the integration.

Some of the embodiments disclosed herein relates to a single-use/disposable plastic based sensor module comprising a at least one transducer element and electrical conductors for electrically connecting between the sensor module and a reusable wearable housing (also referred to herein as monitoring or acquisition device). Optionally, and in some embodiments preferably, several different sensing elements, each comprising one or more transducer elements, are integrated on/in the same sensing module. These different sensing elements can include pressure sensor(s), flow sensor(s), temperature sensor(s), conductivity sensor(s), pH sensor(s), and optical transparency sensor(s). Further sensor elements can be included to measure vibrations and/or acoustic signals (e.g., like a microphone).

Optionally, and in some embodiments preferably, one or more circuitries are also integrated in the sensor module, such as, but not limited to, surface-mount devices (SMD), resistors, memory module(s), application-specific integrated circuit(s) (ASIC), radiofrequency (RF) antenna, energy harvesting module(s), Wi-Fi module(s), Bluetooth low energy (BLE) module(s), battery, and suchlike. Communication with the sensor module can be implemented over electrically conducting wires, and/or wirelessly by RF (or optical e.g., infrared) communication towards external device(s). Alternatively, or additionally, communication with external device(s) can be carried out acoustically through the dispensed fluid (e.g., delivered medicament or other flowing media). Energy harvesting can be used for powering the sensor module and/or its monitoring/acquisition device, which may include using any RF transmitter (e.g., Wi-Fi transmitter, NFC, BLE, Zigbee, LoRa, mobile phone) for a short or a long period of time.

Optionally, and in some embodiments preferably, different connectivity options are utilized for connecting between the wearable housing and the sensor chip, such as, but not limited to, different mechanical connectivity, different electrical and power connectivity (e.g., battery). Different connectivity options can be also used for communicating between the wearable housing and external devices, such as, but not limited to, different mechanical and electrical connectivity, different communication connectivity (e.g., BLE, WiFi, Zigbee, LoRa) and different power supply connectivity (e.g., energy harvesting, wired, battery).

A GPS (global positioning system), and/or localization algorithm, is used in some embodiments, preferably to identify the position of the target to which the fluid dispensed through the sensor module is provided (e.g., a patient in a hospital, home, etc.), which can be integrated inside the wearable housing e.g., implemented as a watch-like wrist-wear device. Optionally, and in some embodiments preferably, the wearable housing/watch-like device can receive and visualize messages or alerts from the medical personnel to the user e.g., patient. Optionally, the user/patient can use the wearable housing/watch-like device to send information or alerts to management authorities e.g., medical care-giving personnel.

In some embodiments additional sensor(s) are integrated in the wearable housing/watch-like device to identify body posture of the user/patient (e.g., standing, lying), and/or measure heartbeat rate, glucose level, oxygen saturation, and suchlike. Optionally, and in some embodiments preferably, a flow control system (e.g., linear piston) is implemented in, and/or remotely controlled by, the wearable housing/watch-like device.

Optionally, disposable drug release elements are integrated inside the wearable housing/watch-like device, comprising: (i) sensor and drug capsule, for slow release of drugs; (ii) sensor with integrated drug release mechanism; and/or (iii) just drug release mechanism.

A passive or active fluid mixer mechanism can be integrated in the wearable housing/watch-like device to add additional drug(s) or other liquid substance(s) from an external source to the delivered/dispensed fluid. The term passive mechanism used herein to refer to a passive structure or mechanism that does not include actuating elements. The term active mechanism is used herein to refer to an active mechanism, e.g., electroactive.

The measurement data/signals from the sensor module(s) can be read by circuitries of the reusable wearable housing/watch-like device, which can be stored in an internal memory and/or transmitted to an external system or reader. The wearable housing/watch-like device can be powered by an internal power source (e.g., battery), external power source (e.g., wired power supply), or using an energy harvesting module which is powered by an external source (e.g., by RF energy).

In some embodiments, the sensor module(s) are connected to fluid supply/dispensing vessels/conduits by plastic tubing using various mechanical connection means e.g., a thread (male or female), a Luer Lock connection (male or female), or a barb connection.

The present application provides in some embodiments for integration of the multiple sensing modules/elements within a single device in a combination of both disposable and non-disposable sensing modules/elements. The properties measured by the sensor module/element includes in some embodiments, but not limited to, fluid pressure, flow rate, electrical conductivity, temperature, pH, transparency, vibrations and/or acoustic signals.

Optionally, and in some embodiments preferably, the sensor module(s) are integrated into the reusable wearable housing/casing or watch-like device in different mechanical ways, such as, but not limited to, a receptacle spring loaded slot or cavity or mechanical clips or magnets, or by mechanical compression by the lid.

Optionally, and in some embodiments preferably, the sensor module(s) are integrated into the reusable casing or watch in different electrical ways, such as, but not limited to, electrical contacts (e.g., pads provided on the sensor module are contacted by pins on the reusable wearable unit) or by contactless means (e.g., NFC reader on the reusable wearable unit, NFC chip with integrated A/D converter and power harvesting units).

For an overview of several example features, process stages, and principles of the invention, the sensor module/apparatus examples illustrated schematically and diagrammatically in the figures are intended for a fluid dispensing applications. These modules/apparatuses are shown as one example implementation that demonstrates a number of features, processes, and principles used to provide fluid measurement setups, but they are also useful for other applications (e.g., that don't dispense fluids) and can be made in different variations. Therefore, this description will proceed with reference to the shown examples, but with the understanding that the invention recited in the claims below can also be implemented in myriad other ways, once the principles are understood from the descriptions, explanations, and drawings herein. All such variations, as well as any other modifications apparent to one of ordinary skill in the art and useful in measurement applications requiring disposable sensor elements may be suitably employed, and are intended to fall within the scope of this disclosure.

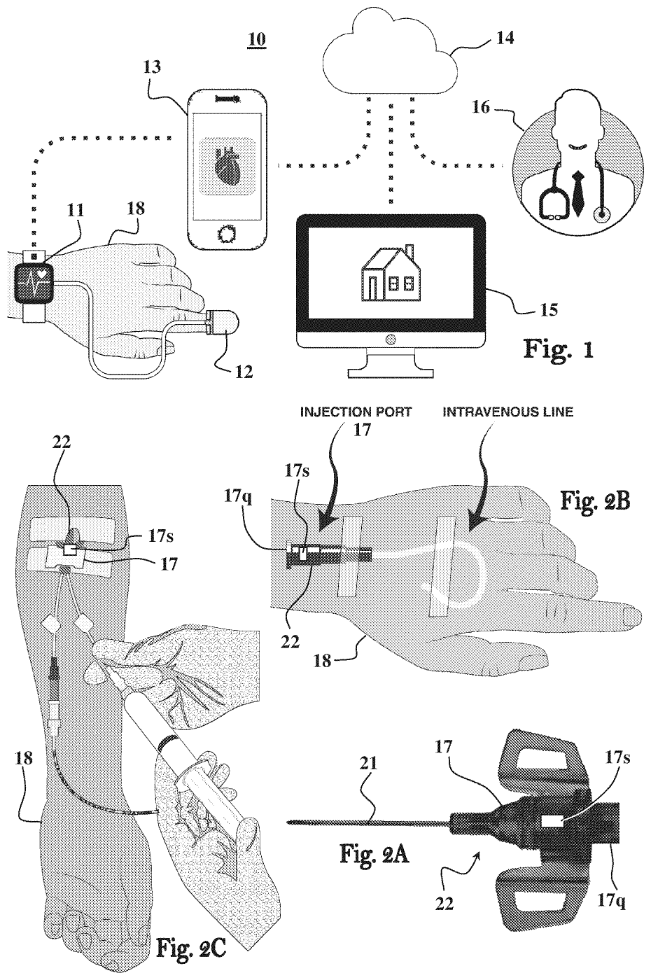

FIG. 1 is a schematic illustration exemplifying concepts of Internet of Medical Things (IoMT) connectivity configuration 10. In this non-limiting example, an external sensor device 12 is wired to a smart watch 11 that retrieves the measurements from the sensor device 12 and transmits the measured data/signals to one or more external devices/computers 13. The external devices/computers 13 can be configured to communicate the data/signals from the sensor device 12 to one or more remote computers/data networks (e.g., the ‘cloud’) 14 for storage and/or processing e.g., using artificial intelligence—AI. As demonstrated, in FIG. 1 care-giving personal (e.g., doctor) and the user/patient 18 at home can retrieve the measurement data/signals from the external devices/computers 14.

FIG. 2A shows a (reusable or single use) fluid dispensing (needle) hub 22 having one or more sensor elements 17s integrated thereinto to provide a sensor module 17 connectable to a needle (e.g., subcutaneous infusion needle) 21. FIGS. 2B and 2C show possible applications wherein fluid dispensing hubs 22 having one or more integrated sensor elements 17s are placed according to the needle access point location (hand or harm 18). This way the sensor chip 17 can be connected to the injection port of any fluid dispensing system. FIG. 2B illustrates a hand access point, in which the fluid dispensing hub 22 with its one or more sensor elements 17s are attached to the wrist of a user/patient 18, and the connector 17q of the sensor chip 17 is readily positioned for connection to a fluid supply tube/line (not shown). FIG. 2C illustrates an arm access point, in which the fluid dispensing hub 22 of the sensor chip 17 is placed as forearm band.

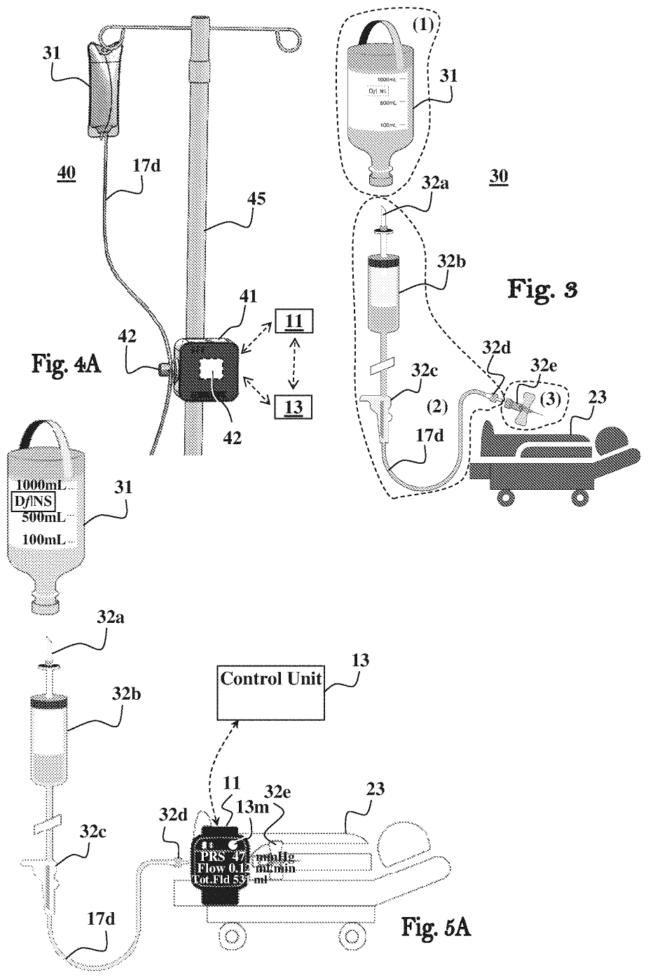

FIG. 3 schematically illustrates a typical IV (intravenous) gravity infusion system 30, which includes three main sterile parts: (1) fluid supply bottle or bag 31; (2) infusion set comprising a spike end 32a for connecting to the fluid supply 31, drip chamber 32b, roller clamp 32c for manual flow control, and connector (e.g., Luer lock) 32d; and (3) IV butterfly/needle 32e connectable to the connector 32d. The main disadvantages of such IV systems 30 are: (i) rough flow control by the manual roller clamp 32b; (ii) lack of flow rate measurement; (iii) Expensive medical staff time wasted in verifying flow integrity; and (iv) low reliability.

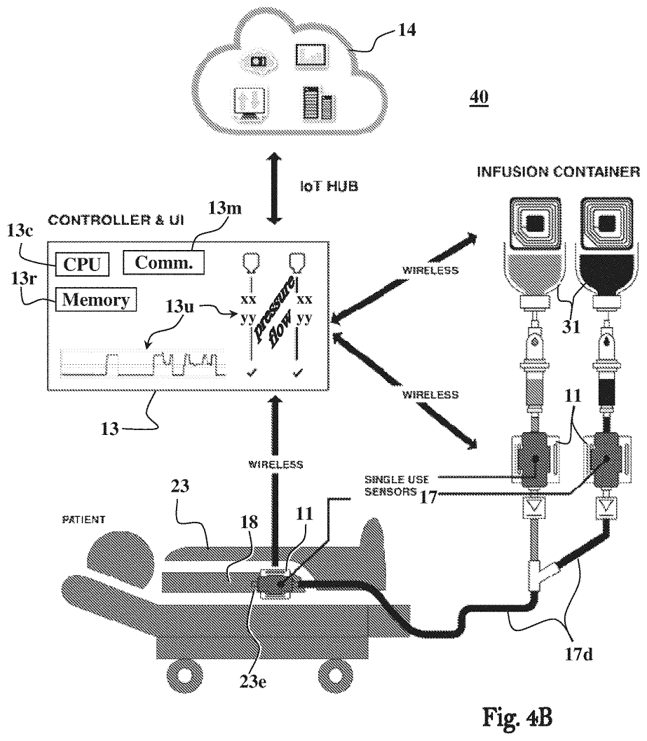

FIG. 4A schematically illustrates a fluid dispensing system 40 comprising a fluid source 31 (e.g., infusion bag) sustained from a support 45 (e.g., infusion pole), a flow control device 41 (e.g., infusion pump) attached to the support 45 and comprising a controlled valve (e.g., proportional pinch valve) 42 coupled to the fluid supply tube/line 17d. The flow control device 41 comprises a data/signals communication unit 42 configured to communicate (over conducting wires and/or wirelessly) data/signals with external devices, such as the wearable unit 11 operatively attached to a user/patient and/or the control unit 13. This way the wearable unit 11, and/or the control unit 13, can be used to control the flow rate of the fluid dispensed through the fluid supply tube/line 17d. For example, if a proportional pinch valve 42 is used, the flow control unit 41 can receive control signals generated by the wearable device 11 and/or the control unit 13 to squeeze (decrease the flow rate) or release (increase the flow rate) the fluid supply line/tube 17d with compliance with the measurement data signals from the wearable unit 11. Optionally, the infusion bag 31 can be pressurized in order to increase the flow rate. While the fluid control device 41 can be attached to the support 45, as exemplified in FIG. 4A, alternatively it can be attached directly to the fluid supply line 17d.

FIG. 4B schematically illustrates a suggested IoMT configuration 40 for infusion systems, which is based on disposable sensor modules 17 that are integrated directly to the line/tube 17d between one or more fluid sources 31 and the entry point 23e to the body of user/patient 23. In this non-limiting example the sensor modules 17 (e.g., for measuring fluid pressure and/or flow rate and/or temperature and/or electrical conductivity and/or optical parameters) are part of the single-use/disposable kit and can be thrown/disposed after usage. However, in this example each reusable wearable housing 11 comprises sockets configured for connecting to the replacement/disposable sensor modules 17.

The reusable wearable housing 11 includes all the required power supply and connectivity modules and circuitries required for carrying out data/signals communication with the control unit 13 over electrically conducting wires, and/or wirelessly. The control unit 13 in this non-limiting example comprises one or more processors 13c and memories 13r configured and operable to store and execute programmed code instructions for operating different functions and procedures of the system, a user interface 13u (UI, e.g., such as of a smartphone, a nurse station or a tablet or a dedicated device) and IoT communication hub 13m configured to communicate with one or more remote computers and/or data networks (e.g., the Internet/cloud and AI system and/or hospital servers and/or patients' Electronic Medical Record) 14. The control unit 13 enables in some embodiments to synchronize between the one or more fluid sources 31 and the entry point device 23e to prevent ADE.

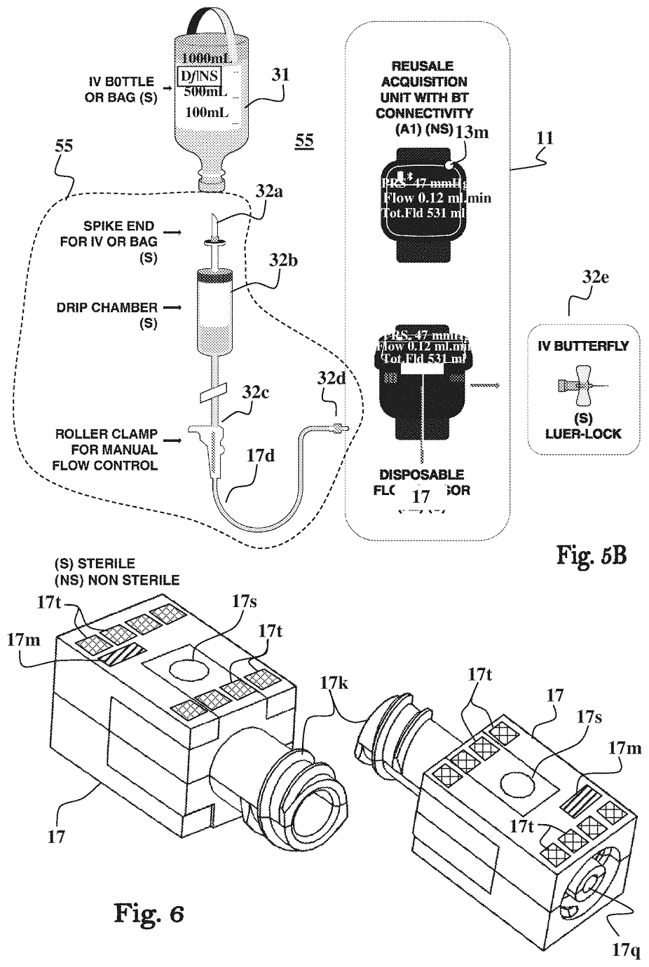

FIGS. 5A and 5B schematically illustrate an acquisition unit 11 having a disposable sensor 17 e.g., for direct flow measurement of fluids dispensed to a user 23 via an infusion system. The disposable sensor 17 is connected to the flow lines 17d of the infusion system between the infusion set 55 and the butterfly needle 32e. The disposable flow sensor 17 is contained inside the reusable and wearable acquisition unit 11. The reusable and wearable acquisition unit 11 comprises electrical contacts and/or circuitries configured to communicate measurement data/signals with the sensor module 17. The sensor module 17 can be configured to measure various parameters/properties of the dispensed fluid media, such as, for example, temperature sensor to measure patient's temperature, electrical conductivity, pH, and/or optical measurements, for example, transparency, to identify a dispensed drug, and/or pressure sensor, for example, to detect disconnection from the patient. These sensors can be further used to prevent ADEs.

In some embodiments the reusable and wearable acquisition unit 11 also comprises wireless communication means (e.g., BLE) for communicating data/signals with one or more external computerized devices/systems e.g., control unit 13. This way the acquisition unit 11 can wirelessly transmit the measurement data/signals generated by the sensor module 17 to the control unit e.g., tablet 13, and/or to another smart computer device). The pairing between the control unit 13 and the acquisition unit 11 can be achieved by near field communication (NFC), for example, to avoid human errors (wrong pairing) and to simplify the pairing procedure in case multiple acquisition units are connected to one tablet 13.

The measurement data/signal generated by the sensor module 17 can be wirelessly transferred, e.g., by WiFi, RF, Zigbee, NFC, or BLE, to one or more remote computers/data networks. For example, in some embodiments WiFi is used to send the measurement data/signal for analysis to the cloud, hospital system, and/or other connected device (e.g., infusion pump/system for flow regulation). Electromagnetic signals can be used for the purpose of data communication with the acquisition unit 11, and/or for energy harvesting. The communication can be done by integrating additional elements in the acquisition device 11 and/or the sensor module 17 e.g., directly on non-deformable portions a diaphragm thereof, such as SMD components, resistors, memory modules, ASIC, RF antenna, energy harvesting modules, Wi-Fi module, BLE module, battery, etc.

FIG. 6 schematically illustrates a disposable sensor module/chip 17 according to some possible embodiments. in this non-limiting example the sensor module 17 comprises fluid delivery connectors e.g., Luer lock (male and female), 17k and 17q, integrated in its main body structure, electrical contact pads 17t configured to electrically connect between one or more sensor elements 17s of the sensor module 17 and the reusable wearable device (11), internal fluidic channels (not show) fluidly communicating between the fluid delivery connectors 17k and 17q, and one or more circuitries 17m e.g., SMD device (e.g., memory chip for storing calibration data, lot number, unique ID of the sensor module, sensor usage time to avoid re-use and over-use, first time of usage, last time of usage). Values/data stored in a memory (17m) of the disposable sensor module/chip 17 can be retrieved and used by the wearable reusable unit 11 during its operation. The one or more sensor elements 17s are implemented in some embodiments by transducing elements formed on a deformable element/diaphragm configured to interact with the fluid media streamed through sensor module, and thereby measure one or more properties thereof.

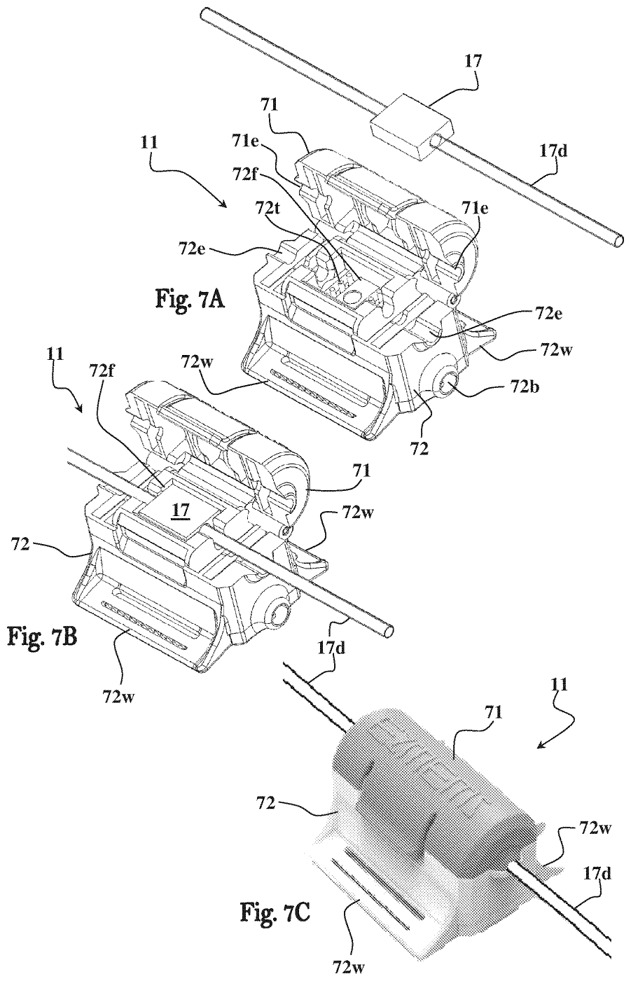

FIGS. 7A to 7C schematically illustrate a reusable wearable housing/device 11 according to some possible embodiments. The reusable wearable device 11 in this non-limiting example comprises a base 72 having one or more sockets 72f (also referred to herein as attachment assembly, only one socket is shown) configured to receive and hold the sensor module 17, and a hinged lid 71 configured to change between open and closed states. FIG. 7A shows the reusable wearable device 11 in the open state before the sensor module 17 is placed in the socket 72f, FIG. 7B shows the reusable wearable device 11 in the open state after the sensor module 17 is placed in the socket 72f, and FIG. 7C shows the reusable wearable device 11 in the closed state after the sensor module 17 is placed in the socket 72f and the hinged lid 71 is closed thereover.

In some embodiments the socket 72f comprises one or more electrical contacts 72t, each configured to establish electrical contact with a respective contact pad (17t) of the sensor module 17 when it is placed thereinside. As seen, the sensor chip/module 17 can be first connected to fluid supply/dispensing line/tubes 17d, and thereafter placed in the socket 72f. The base 72 and the lid 71 comprise open channels, 72e and 71e respectively, configured to receive and hold the fluid lines/tubes 17d connected to the inlet and outlet ports of the sensor module 17, and form closed fastening channels in the closed state of the lid 71 configured to receive and hold the fluid inlet and outlet ports of the sensor module 17 and/or the fluid lines/tubes 17d (e.g., fluid supply/dispensing vessels/conduits). Optionally, the base 72 can further comprise a power socket 72b configured to receive and electrically connect to a power cable for providing external electrical power supply to the sensor module 17.

As seen in FIG. 7C, in the closed state the fluid supply and dispensing tubes 17d extend in sideway directions from the wearable housing/device 11. The base 72 of the wearable housing/device 11 can be releasably attached, or tied, to the user/patient (23), or to other surfaces or equipment e.g., by attachment wings 72w. The hinged lid 71 can be opened whenever needed for placing and/or replacing the sensor chip/module 17. The base 72 receives the sensor chip/module 17 in its socket 72f and configured to provide a wired and/or wireless interface to the sensor elements (17s) of the sensor chip/module 17. In some embodiments the base 72 comprises all the reusable elements and electronic circuitries (e.g., data communication module) required for interfacing with the sensor elements (17s) of the sensor chip/module 17. The lid 71 may also be used to push and hold the sensor chip/module 17 in its position.

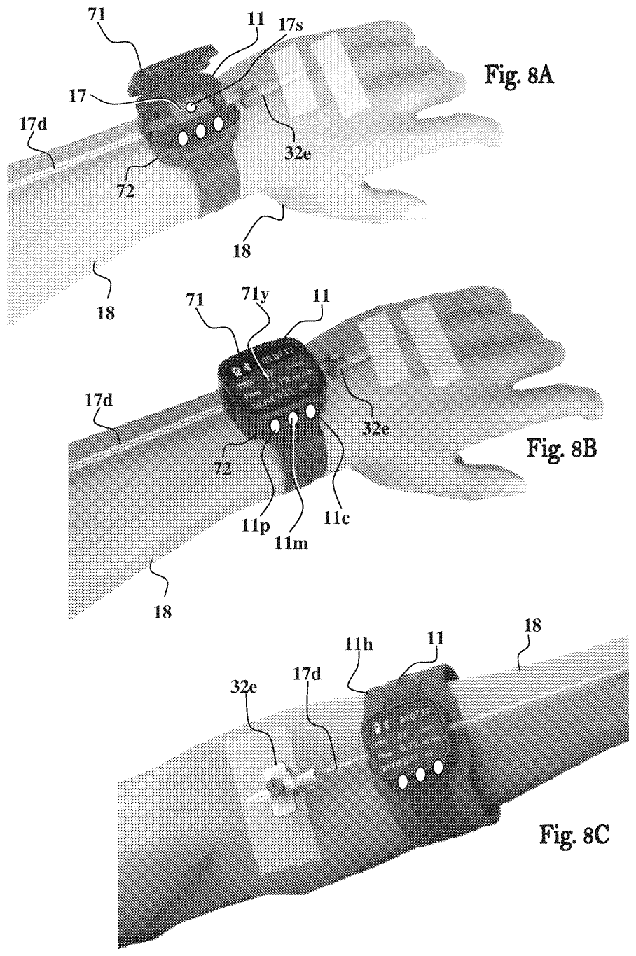

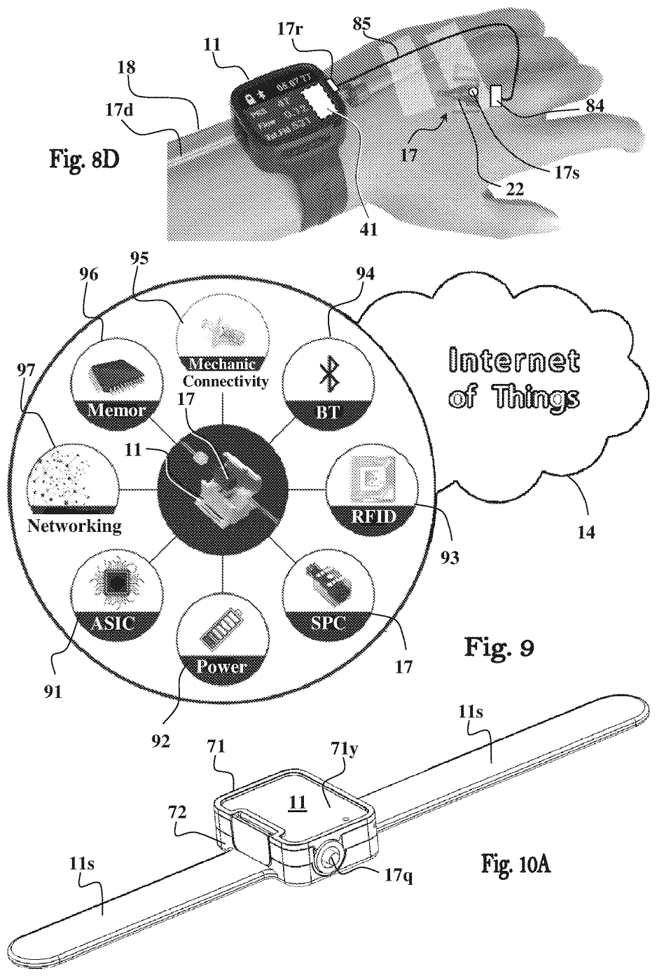

FIGS. 8A to 8C schematically illustrate possible usage examples according to some possible embodiments, wherein fluid properties sensing is integrated inside a smart watch device 11, and the dispensed fluid delivered to the user/patient 18 is passing through the sensor chip/module 17 placed inside the smart watch device 11. The sensor chip/module 17 can be replaceable, and accordingly can be removably mounted inside the reusable wearable hosing 11, which in this case is implemented as a smart watch. FIG. 8A shows the sensor chip/module 17 placed inside the smart watch 11 when the lid 71 is in an open state. FIG. 8B shows the smart watch 11 when the sensor chip/module 17 is held by the socket (72f) inside and the smart watch 11 and the lid 71 is closed.

In this specific and non-limiting example, the wearable housing/device 11 is also used as a monitoring device. The lid 71 thus comprises in some embodiments a display 71y (e.g., a smart watch display) used to show data indicative of the measurement data/signals generate by the sensor elements 17s, such as measured fluid pressure, fluid temperature, fluid transparency, total flow amount, patient's temperature and heartbeat, flow rate, and other possible parameters, and also regular data-display of the watch itself, such as the current time, battery level, status of wireless connectivity. Accordingly, the wearable housing/device 11 comprises in some embodiments one or more processors 11p and memories 11m configured to store and execute program code instructions for processing the data/signals generated by the sensor elements 17s of the sensor module 11 and displaying corresponding information in the display 71y of the smart watch. For example, a chip with edge computing and AI capabilities can be integrated into the wearable housing/device 11 for carrying out data/signals analysis procedures.

In some embodiments the wearable device/housing 11 further comprises a communication module 11e configured to exchange data/signals with a remote computer and/or data network. For example, the wearable device/housing 11 can be configured to transmit data/signals indicative of the measured properties/conditions of the dispensed fluid and/or of the user/patient 18, and/or receive control data/signals and/or instructions from the remote computer/network e.g., to adjust, or stop, flow rate of the dispensed fluid. Though the data/signals communication with the wearable device/housing 11 can be carried over serial/parallel communication lines/bus (e.g., USB, UART), in preferred embodiments wireless communication is used (e.g., using WiFi, Bluetooth, BLE, ZigBee, or suchlike).

FIG. 8C shows an additional configuration utilizing the smart watch 11 with an elastic stretch 11h, and a different entrance point to the body 18 of the user/patient.

FIG. 8D shows another possible application wherein the wearable device/housing 11 is used to interface with the sensors elements 17s of the dispensing hub 22 shown in FIGS. 2A to 2C. In this non-limiting example, the wearable device/housing 11 is connected to the sensor module 17 integrated into the dispensing hub 22 by one or more electrically conducting wires 85 that are connected at one end thereof to an electrical connector 17r of the wearable device/housing 11. An electrical connector 84 is provided at the other end of the conducting wires 85 for connecting to the sensor module 17. Alternatively, the communication between the sensor module 17 and the wearable device/housing 11 is conducted wirelessly.

In this non-limiting example, there is no need for a sensor module 17 inside the wearable device/housing 11. Accordingly, the wearable device/housing 11 is used to anchor the fluid supply tube/line 17d to the arms 18 of the user, for displaying information associated with the measurement data/signals generated by the one or more sensor elements 17s, and/or for communicating measurement the data/signals to external devices (over conducting lines and/or wirelessly). In some embodiments, a flow control device 41 (such as shown in FIG. 4A) is placed inside wearable device/housing 11 for controllably regulating the flow of the dispensed fluid e.g., based on the data/signals generated by the one or more sensor elements 17s.

FIG. 9 schematically illustrates different connectivity options of the wearable device/housing 11 according to some possible embodiments, such as, but not limited to, different mechanical connectivity to the sensor chip/module (networking 97, memory device 96, mechanical connectivity 95, ASIC 96), different communication connectivity (Bluetooth—BT 94, RFID 93) and different power connectivity (e.g., battery 92), possibly used to communicate data/signals with the sensor chip/module 17.

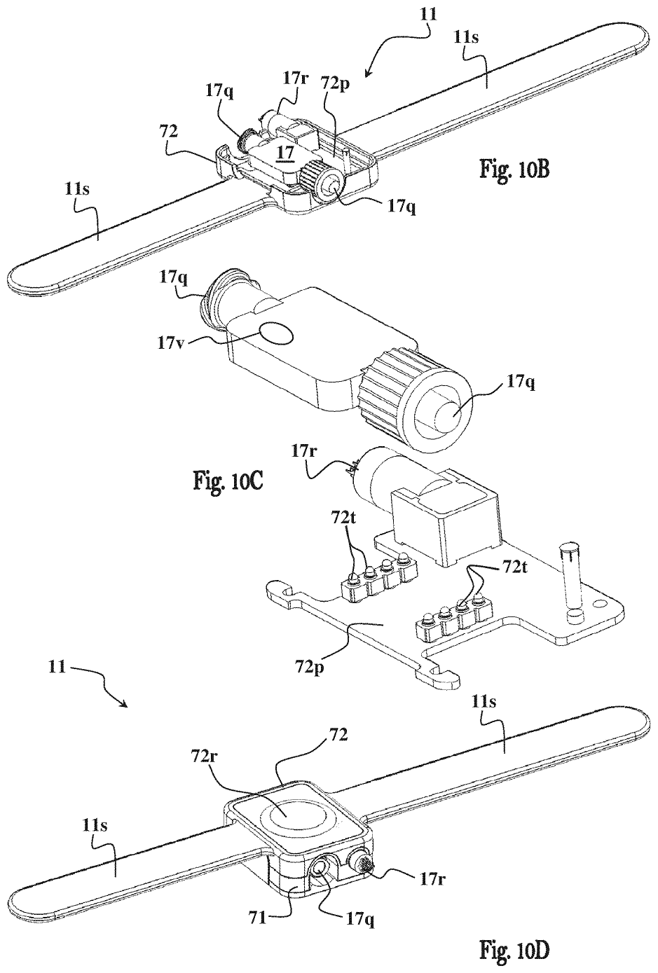

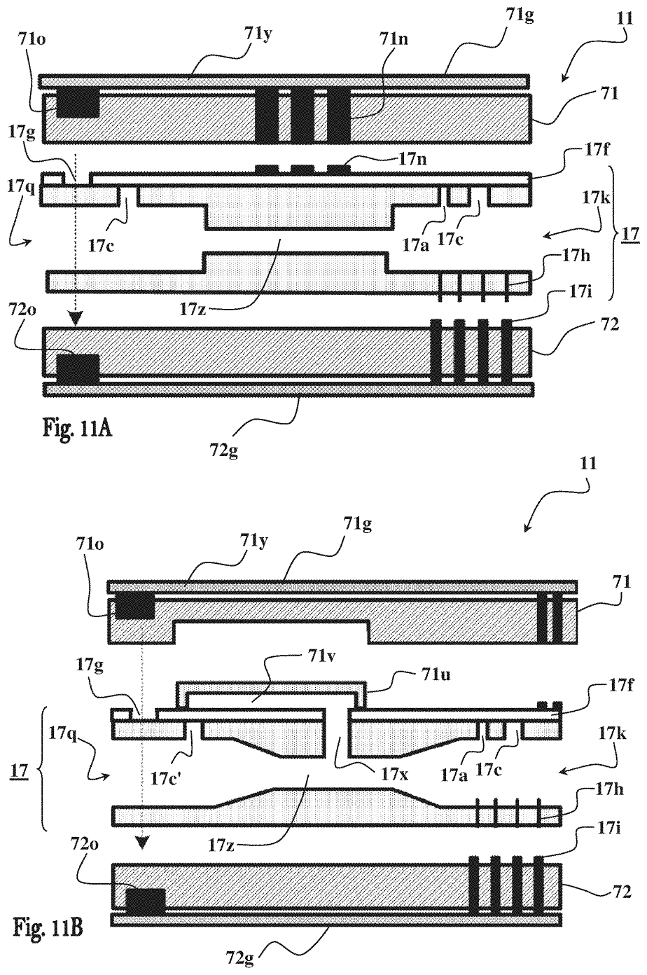

FIGS. 10A to 10D schematically illustrate the smart watch device 11 according to some possible embodiments. FIG. 10A shows the smart watch 11 with the lid 71 in the closed state, the display 71y in an operative state, the sensor module (17) placed in the base 72 without fluid connection to supply/dispensing tubes/lines, and with its straps 11s extending in side way directions. FIG. 10B shows the smart watch device 11 containing the sensor module 17 and the lid (71) removed. As seen, in this non-limiting example the base 72 is configured to accommodate the sensor module 17, a circuit board 72p (e.g., PCB) configured to interface to the sensor module 17, and an electrical connector 17r for connecting the smart watch 11 to electrical supply/charger and/or for communicating data signals therewith e.g., as exemplified in FIG. 8D.

FIG. 10C shows an exploded view of the printed circuit board 72p and the sensor module 17. As seen, the printed circuit 72p can have one or more electrical contractors 72t (eight contactors 72t are shown in FIG. 10C) configured to electrically connect to the contact pads (17t in FIG. 6) of the sensor module 17. FIG. 10D shows a bottom side of the smart watch device 11 in the closed state with the sensor module (17) placed thereinside. In some embodiments the smart watch 11 comprises one or more sensing elements 72r mounted at the bottom side of the smart watch device 11 and configured to contact the skin of the user/patient and measure various parameters thereof e.g., body temperature, heartbeat rate, blood pressure. As exemplified in FIG. 10C, in some embodiments the sensor module comprises a drug release capsule 17v e.g., adapted for slow release of a medicaments into the dispensed fluid media.

FIGS. 11A and 11b show sectional views showing the interface of the wearable acquisition/monitoring device 11 according to some possible embodiments. In these non-limiting examples, the lid 71 comprises lid circuitries 71g mounted thereon e.g., incorporating, or carrying, the display 71y. As shown, the sensor module 17 may comprise electrodes 17h configured to contact the fluid dispensed through the sensor module 17 and measure one or more electrical properties (e.g., electrical conductivity, resistance, dielectric constant, and/or dissipation factor) of the dispensed fluid. In such embodiments the base 72 may comprise base circuitries 72g mounted therebeneath and configured to electrically contact the electrodes 17h via contact pins 17h passing through the base 72 from its top side to its bottom side. The sensor module 17 can further comprise a deformable element e.g., thin film or foil 17f attached over a section of the body of the sensor module 17 and having one or more transducing elements formed thereon or therein for measuring one or more properties of the dispensed fluid.

In this non-limiting example, the sensor modules 17 are adapted to conduct optical measurements for determining one or more optical properties of the dispensed fluid e.g., reflectance, transmission, opacity, transparency, which may be used to determine turbidity/transparency of the dispensed fluid. In some embodiments the lid circuitries 71g comprises a one or more light sources e.g., LEDs 71o, configure to radiate light into the fluid passage of the sensor module 17, and the base circuitries 72 comprises respective one or more light detectors 72o configured to receive the light transmitted through the dispensed fluid and measure its intensity. For this purpose one or more openings 17g can be formed in the deformable element 17f for the radiated light to pass into the fluid passage of the sensor module, and the lid 71 and the base 72, or at least some portion thereof, are made transparent for the same purpose. The casing of the acquisition/monitoring device (watch) 11 can be designed to avoid entry of interfering environmental light towards the optical measurement paths defined between the one or more light sources 710 and their respective one or more light detectors 72o, to thereby prevent influence/disturbance to the optical measurements.

As also seen, the fluid passage of the sensor module 17 may comprise a constriction 17z configured to cause a pressure difference between its fluid supply and fluid dispensing cavities (shown at 17q and 17k). In order to conduct differential pressure measurements the sensor module 17 comprises openings 17c at the fluid supply and fluid dispensing cavities which are configured to allow the fluid passed through the sensor module 17 to interact with portions of the deformable element 17f on which transducing elements ae formed for measuring strains thereby imparted. The measured strains can be then processed to determine the differential pressure and therefrom the flow rate of the dispensed fluid media. In some embodiments an additional opening 17a is provided at the fluid supply and/or fluid dispensing cavities, to enable a respective portion of deformable element 17f carrying a transducing element configured to measure temperature to interact with the dispenses fluid.

In FIG. 11B an auxiliary opening 17x is formed in the constriction 17z passing through the body of the sensor module 17 and the deformable element 17f for communicating fluid media from the constriction 17z into an auxiliary cavity 71v formed over a portion of the deformable element 17f, and thereby cause a Venturi effect. The auxiliary cavity 71v can be formed by a cup member 71u sealably attached over a portion of the deformable element 17f, over the auxiliary opening 17x, and over the opening 17c′, to thereby use the Venturi effect for measuring the pressure drop from the entrance to center of the restrictor (throat) 17z utilizing transducing elements formed in the portions of the deformable element 17f covering the opening 17c′.

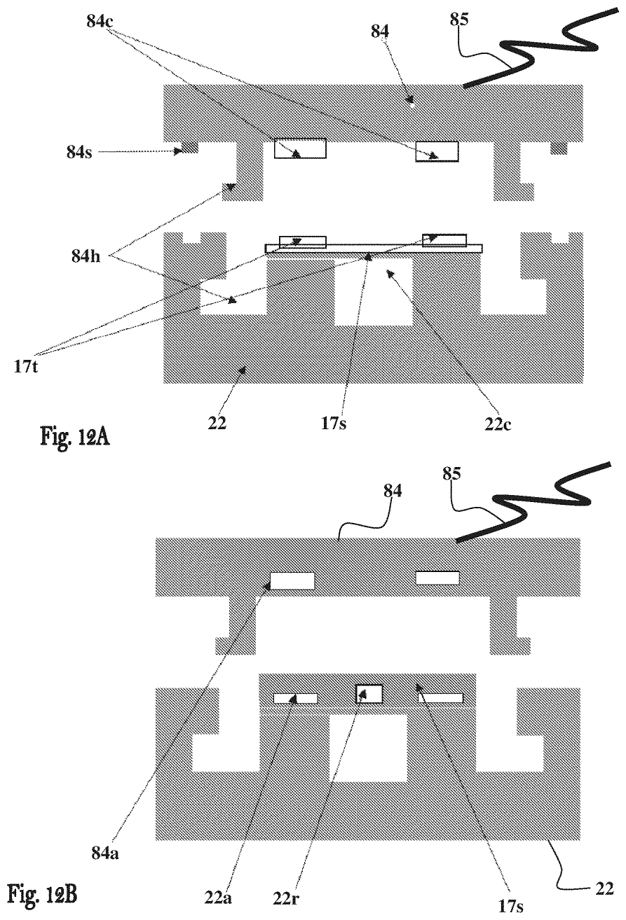

FIGS. 12A and 12B schematically illustrates possible designs of the connector 84 used to connect to the dispensing hub 22 sensor module shown in FIG. 8D. In these non-limiting examples a latching mechanism 84h is used to establish mechanical connection between the connector 84 and the dispensing hub 22, and the one or more sensor elements 17s carrying transducing elements are configured to interact with fluid media dispensed through the fluid channel 22c of the dispensing hub 22. In FIG. 12A electrical contacts (e.g., spring load pins) 84c, electrically connected to the conducting line 85, are used at the bottom side of the connector 84 to electrically connect to the contact pads 17t of the dispensing hub sensor module 22. Optionally, sealing means 84s also provided at the bottom side of the connector 84 for water protection.

FIG. 12B schematically illustrates a wireless embodiments wherein the connector 84 comprises one or more antenna elements 84a configured for data/signals communication and/or power supply for the dispensing hub sensor module 22 (e.g., using NFC communication and energy harvesting). In this non-limiting example, the dispensing hub sensor module 22 also comprises one or more antenna elements 22a for data/signals communication with the wearable/acquisition device (11) and/or for self-power supply by means of the energy harvesting circuitries 22r also provided therein. The energy harvesting circuitries 22r can also include communications, analog front-end and analog to digital data conversion circuitries. The sensor element 17s in this non-limiting example is provided with a sealed, such that there is no need for additional sealing between the connector 84 and the dispensing hub sensor module 22.

Terms such as top, bottom, front, back, right, and left and similar adjectives in relation to orientation of the devices/elements and components thereof refer to the manner in which the illustrations are positioned on the paper, not as any limitation to the orientations in which the apparatus can be used in actual applications.

As described hereinabove and shown in the associated figures, the present invention provides sensor devices/modules for measuring properties of dispensed fluids, and related methods. While particular embodiments of the invention have been described, it will be understood, however, that the invention is not limited thereto, since modifications may be made by those skilled in the art, particularly in light of the foregoing teachings. As will be appreciated by the skilled person, the invention can be carried out in a great variety of ways, employing more than one technique from those described above, all without exceeding the scope of the claims.

1. A monitoring device comprising:
housing configured to attach to a user or to a user aid item;
at least one attachment assembly provided in said housing and configured to attach to a respective at least one removable sensor module;
electric circuitries or contacts mounted in or on said housing and configured to provide an electrical interface between said at least one removable sensor module and one or more external devices; and
inlet and outlet fasteners provide in said housing and configured to receive and hold respective fluid supply and fluid dispensing lines, said fluid supply and fluid dispensing lines being connected to said at least one removable sensor module attached to said at least attachment assembly for dispensing fluid media therethrough,
said at least one removable sensor module configured to measure at least one property or condition of said fluid media, and said electrical circuitry configured to communicate measurement data or signals generated by said at least one sensor module to said one or more external devices.

2. The monitoring device of claim 1 wherein the electric circuitries are configured to wirelessly communicate the measurement data or signals to the one or more external devices.

3. The monitoring device of claim 1 having one or more processors configured and operable to process the measurement data or signals generated by the at least one removable sensor module and generate control data or instructions for a flow control unit for controlling flow rate of the fluid media.

4. The monitoring device of claim 3 comprising a display device, and wherein the one or more processors are configured to display in said display device information associated with the measurement data or signals.

5. The monitoring device of claim 1 wherein the fluid media comprises a medicament dispensed into the user.

6. The monitoring device claim 1 wherein at least one of the removable sensor modules comprises electrodes configured to contact the dispensed fluid media and measure one or more electrical properties thereof.

7. The monitoring device of claim 1 wherein the at least one removable sensor modules is configured to measure one or more of the following: fluid pressure; flow rate; temperature of the dispensed fluid media and/or of the user; electrical conductivity and/or resistance, dielectric constant and/or dissipation factor; pH level; optical transparency and/or transmission; and/or acoustic signals transferred through the dispensed fluid media.

8. The monitoring device of claim 7 wherein the control unit is configured and operable to process the measurement data/signals from the at least one removable sensor module and identify based thereon one or more substances comprised in the dispensed fluid media.

9. The monitoring device of claim 1 wherein the housing is configured as a wrist-wear device.

10. A measurement arrangement comprising: the monitoring device of claim 1; and
the at least one removable sensor module removably attached to the attachment assembly, at least one of said removable sensor modules comprises at least one sensor element made of polymeric or plastic materials and having one more transducing elements formed thereon or therein.

11. The measurement arrangement of claim 10 wherein at least one of the removable sensor modules is a disposable sensor module.

12. The measurement arrangement of claim 10 wherein the at least one sensor element comprises electrical contacts electrically connected to the one more transducing elements are formed on, or at least partially in, a deformable element.

13. The measurement arrangement of claim 12 wherein the deformable element is made of an integrated multilayered foil or film.

14. The measurement arrangement of claim 13 wherein at least one external layer of the multilayered foil or film is configured to enable attachment to an injected molded plastic for assembly in a body of the sensor module.

15. The measurement arrangement of claim 10 wherein the one or more transducing elements are configured to form at least one differential pressure measurement circuitry.

16. The measurement arrangement of claim 10 wherein the housing and/or at least one of the one or more sensor modules comprises optical elements configure to measure at least one optical property of the dispensed fluid media.

17. The measurement arrangement of claim 10 comprising a flow control device configured to controllably adjust flow rate of the dispensed fluid media.

18. The measurement arrangement of claim 17 wherein the control unit is configured to generate control signals for adjusting the flow rate of the dispensed fluid media based on the measurement data/signals from at least one of the removable sensor modules.

19. A method of measuring one or more properties of a dispensed fluid media, the method comprising:
connecting at least one fluid supply conduit to inlet port of a respective at least one sensor module, said at least sensor module having a fluid passage in fluid communication with said inlet port and one or more sensor elements configured to measure one or more properties of fluid media in said fluid passage;
coupling between said at least one sensor module and an acquisition unit for communication of data/signals therebetween, said acquisition unit configured to provide an interface to said at least one sensor module; and
processing data/signals generated by said at least one sensor module and obtained via said acquisition unit.

20. The method of claim 19 comprising at least one of the following: communicating the measurement data/signals from the acquisition unit to at least one external device; generating control signals/data based on the measurement data/signals for regulating flow of the dispensed fluid media; processing the measurement data/signals and identifying at least one substance contained in the dispensed fluid media; and attaching the at least one sensor module to the acquisition unit or to a user receiving the fluid media.

21-23. (canceled)

24. The monitoring device of claim 1 wherein the electric circuitries comprising at least one of the following: an energizing module configured to energize a harvesting module of the at least one removable sensor module, data/signals communication conductors for communicating data/signals with the at least one removable sensor module, and/or a wireless data communication module for communicating data/signals with the at least one removable sensor module.
